(19) United States
(12) Patent Application Publication
(10) Pub. No.: US 2006/0160157 A1
(43) Pub. Date: Jul. 20, 2006

(54) Method, compositions and classification for tumor diagnostics and treatment

(76) Inventor: Mathew Mark Zuckerman, Woody Creek, CO (US)

Correspondence Address: MCHALE & SLAVIN, P.A., 2855 PGA BLVD, PALM BEACH GARDENS, FL, 33410, US

(21) Appl. No.: 11/334,845

(22) Filed: Jan. 19, 2006

Related U.S. Application Data
(60) Provisional application No. 60/645,077, filed on Jan. 19, 2005.

(51) Int. Cl. G01N 33/574; C07H 21/04; C12P 21/06; C07K 14/82; C07K 16/30; C07K 16/46; A61K 51/00

(52) U.S. Cl. 435/7.23; 435/69.1; 435/320.1; 435/325; 530/350; 530/388.22; 536/23.5; 424/1.49

(57) ABSTRACT

The present invention is directed towards classifying tumor biomarkers, particularly membrane receptors, and more particularly the gastrin-releasing peptide (GPR) receptors, identified in patient samples, then linking therapeutic agents (chemical, radiological, or biological) to patient-specific ligands that bind to such receptors, clinicians can produce diagnostic and treatment compositions and implement treatment regimens which, by using the classified and identified biomarkers, and due to their improved accuracy, increase success and decrease undesired side effects from such treatments.

### REFERENCE TO RELATED APPLICATIONS

This application claims the benefit of the provisional patent application 60/645,077 titled “Methods, Compositions, and Diagnostics for Classifying Tumors and Reducing Tumor Mass”, filed on Jan. 19, 2005 by the same inventor, the contents of which are herein incorporated by reference in their entirety.

### FIELD OF THE INVENTION

This invention relates to a method that enables identification and separation of ligands (proteins) that are specific to a cancerous cell and to at least a particular patient. It particularly relates to diagnostic and/or therapeutic applications, wherein the ligands can, after separation, be replicated and tagged with either a radioactive agent for the SPECT and/or PET tracer or a fluorescence (dye) for a Receptor TRAP. The invention most particularly relates to a technology platform which is based on gastrin releasing peptide receptors (GRP-R), which are useful as biomarkers for a number of cancer forms including renal cancer, prostate cancer and lung cancer.

### BACKGROUND OF THE INVENTION

Current methods of diagnosing and treating cancers are, for the most part, based on the concept of “organ of origin”, i.e. breast, prostate, lung and other organs. Such a methodology seeks to diagnose, classify, and treat a tumor in a patient by first determining the organ in which the tumor is found, or is known or believed to have originally developed. The spatial area(s) of greatest density are attacked as the physicians seek to eliminate or reduce a patient's tumor while sparing non-cancerous tissues.

It is known that expression of, and/or over-expression of, certain cellular proteins, particularly extracellular cell membrane-bound receptors, are hallmarks of cancerous cells. The present inventor has therefore conceived of a paradigm shift in cancer care based upon the premise that certain biomarkers serve as a better predictor of tumor presence and progression than the size of the tumor in the “organ of origin”, and therefore provide a unique approach to the use of cancer cell receptors and biomarkers in diagnostic/therapeutic oncology applications.

The premise here is that the tissue or cell type of origin of a tumor, along with the presence or absence of certain biomarkers, may serve as a better predictor of tumor progression and patient prognosis than the organ of origin or residence of the tumor. It is now recognized that one or more cell growth factor receptors may be aberrantly expressed and/or over-expressed by a number of otherwise disparate types of tumor cells. Thus, for example, epidermal growth factor (EGF) is a peptide growth hormone that stimulates growth of epidermal cells via activation of the epidermal growth factor receptor (EGFR), a transmembrane receptor. EGFR is also expressed by a wide variety of tumor cells, including non-small-cell lung carcinoma, renal cell carcinoma, breast tumor cells, and tumor cells of colorectal origin. Similarly, vascular endothelial growth factor receptor-1 (VEGFR-1 or flt-1) is expressed by a number of tumor cells, including among others cells of breast, colorectal, and other origins, as well as by cells of the tumor vasculature.

Gastrin-reducing peptide (GRP) was originally identified as a mammalian analog of the amphibian peptide, bombesin. GRP has been shown to produce a variety of physiological effects, including growth of normal and neoplastic tissues, smooth muscle contraction, secretion, thermoregulation, circadian rhythm, satiety, and immunological responses. These effects are thought to be mediated via GRP receptors (GRP-R) found throughout the central nervous system and peripheral tissues, as further described below. GRP-R are part of the bombesin receptor family, which includes, in addition to GRP-R, the neuromedin B receptor (NMBR), bombesin receptor subtype 3 (BRS-3), and bombesin receptor subtype 4 (BRS-4).

GRP-R and NMBR are also present on a number of tumor types, particularly those of neuroendocrine origin, such as certain human small-cell lung carcinoma tumors, prostate, and breast tumors. In one survey, it was found that tumor cells from prostate, breast, renal and small-cell lung carcinomas expressed GRP-R and not NMBR, while thymic tumor cells expressed only NMBR. Tumor cells from a gastrinoma and from bronchial tumor expressed both types of receptor. (Reubi, et al., Clin. Cancer Res. 8: 1139-1146, 2002).

While it is now apparent that different tumor types share common tumor receptor biomarkers, a more recent development is the realization that, within these tumor receptor biomarkers, there can be a fairly wide diversity of molecular characteristics. Such variations within each of these tumor receptor sub-types may also result in associated variable ligand binding characteristics among the receptor variants.

Current tumor diagnostic methods rely on gross or overt anatomical abnormalities detected by spatially comparative physical imaging means, ranging from tactile examination to X-Rays, computerized tomography (CT) or magnetic resonance imagine (MRI). Positron emission tomography (PET) using radiolabeled 2-deoxyglucose to detect variations in a general location of specific cellular activity is a more recent addition; but, while this latter can putatively distinguish tumorous tissue, this is also a non-specific indicator of presumptive cancerous tissue, i.e. it cannot necessarily identify benign or irregular tissues. The broader or more diffuse the potential location(s) for the tumorous tissues, the more time and effort must be spent locating and then studying and identifying the tumor - and the greater the incidence of either false positives or operator errors.

It would be useful, therefore, to provide a method for enhancing specificity of the tumor diagnostic process, while simultaneously providing a therapeutic modality for providing enhanced treatment specific to the nature of the tumor and individual patient.

### SUMMARY OF THE INVENTION

Earlier and more accurate detection of cancer has the potential to increase survival and reduce overall healthcare cost affiliated with cancer diagnosis. The present invention provides several diagnostic tools that allow earlier and more accurate detection of cancer. These tools utilize a group of tumor targeted patient specific ligands (PSL), along with radiopharmaceutical agents, to provide enhanced nuclear medical imaging. In the form of a diagnostic kit, or “Receptor TRAP”, these diagnostic tools will provide physicians with an easy and quick determination of the presence of certain cancers from a simple tissue, plasma, whole blood, or urine test. An exemplary, albeit non-limiting example is a technetium based oncology tracer for single photon emission computed tomography (SPECT). Data gathered from use of the SPECT tracer will enablement development of tracers for positron emission tomography (PET). As an oncology specific tracer, the combination of the PSL and PET tracer will enable specificity of PET imaging not heretofore possible. Because many diseases, particularly cancer, “target” the body's healthy cells resulting in abnormal cell behavior such as rapid growth or cell splitting, a cell-specific and, more importantly, patient-specific approach to cancer diagnosis and treatment will provide a crucial new and improved methodology for cancer diagnostics and therapeutics.

Although not wanting to be limited thereto, the instant invention is particularly illustrated with respect to the use of the gastrin releasing peptide receptors described further herein. GRP-R are found only in a few isolated tissue types in normal, healthy individuals. The steps of introducing, letting circulate through normal biological processes, and then localizing densities of GRP-R-specific diagnostic materials outside these tissues will provide a higher level of diagnostic specificity than is otherwise currently available.

More specifically, the invention is directed to methods of classifying GPR tumor receptor subtypes, based on certain physicochemical parameters of the GPR tumor receptor. Such parameters include but are not limited to the ability to bind specific ligands (ligand specificity). Such a profile can then be compared to archival, normal, and synthetic GRP-R subtypes for purposes of classifying the patient's tumor receptor subtype.

Specific tumor receptors of interest are the EGF receptor, the vascular endothelial growth factor (VEGF) receptor, and the bombesin-related receptors, gastrin releasing peptide (GRP) receptor and neuromedin receptor. However, it is appreciated that compositions and methods of the invention may be used with a wide variety of tumor receptors and receptor subtypes.

In a specific, albeit non-limiting embodiment, receptor-specific ligands can be selected from peptide phage display libraries. In a related embodiment, individual members of subgroups of such phage display libraries may be used in compositions, by conjugating to a peptide-bearing filamentous phage diagnostic moiety (or to multiple such moieties), such as radioactive tags suitable for positron emission tomography (PET) scanning and SPECT, or to tumor ablative compounds, such as chemotherapeutics, or to radioactive therapeutic substances.

In a related embodiment, the foregoing diagnostic and therapeutic methods and compositions may be brought together in an environment conducive to individualized patient, or even tumor, diagnosis and treatment. According to this feature of the invention, tumor receptors are harvested from patient tumor biopsy or serum samples and are classified to provide a tumor receptor subtype profile, based on the classification schemes described herein, and such classification is used to make individualized and targetable diagnostic and therapeutic agents.

Accordingly, it is an objective of the present invention to identify and isolate tumor receptor biomarkers common to a diversity of tumor cell types, and provide diagnostic and therapeutic methods, compositions, and supportives that take advantage of these identifiably distinct molecules as diagnostic and treatment targets, in order to both individualize treatment regimens and target them at an individual's tumor receptor subtype, independent of the known or suspected “organ of origin”.

It is a further objective of the instant invention to provide diagnostic and therapeutic regimens that take advantage of the common features among tumors diverse in origins, locations, current status, or patients, in order to provide an economical and expedient way to diagnose and treat tumors.

It is yet another objective of the instant invention to provide methods and compositions that exploit the tumor receptor biomarker similarities to aid classification across tumors of diverse origins, by optimizing the isolation of ligands that bind to such receptor subtypes.

It is a still further objective of the present invention is teach methods, compositions, and diagnostics for classifying tumor receptor biomarkers, generally cellular membrane receptors, in individual patients and in each of those same individual patient's tumor, tissue, and serum samples.

It is yet another objective of the instant invention to teach a method for determining the presence (or absence) of a particular receptor subtype on the tumorous cell's active tissue.

It is a still further objective of the instant invention to utilize the foregoing classification methods to create diagnostic and therapeutic compositions suitable for further, more precise diagnosis and treatment of tumors.

These and other features of the invention will become more fully apparent when the following detailed description of the invention is read in conjunction with the accompanying drawings.

### Definition of Terms

Terms utilized throughout this application are understood to embrace the following meanings:

Agonist: A moiety which binds to a receptor of a cell thereby triggering a response by the cell. An agonist produces an action. It is the opposite of an antagonist which acts against and blocks an action.

Antagonist: A moiety which acts against and blocks an action. For example, insulin lowers the level of glucose (sugar) in the blood, whereas another hormone called glucagon raises it; therefore, insulin and glucagon are antagonists. An antagonist is the opposite of an agonist which stimulates an action.

Ligand: A molecule that binds to another. Often, a soluble molecule such as a hormone or neurotransmitter that binds to a receptor.

Indigenous Ligand—a naturally occurring protein, which may be an agonist or an antagonist, and which binds to a particular receptor type in mammals.

Tumor Receptor Specific Ligands—a plurality of proteins associated with and including said indigenous ligand and characterized as having different sequences but the same number of amino acids as the indigenous ligand, and which bind to aberrantly expressed receptors on cancerous cells with varying degrees of specificity and/or affinity.

Patient Specific Tumor Receptor Ligands—a select group of Tumor Receptor Specific Ligands exceeding a minimum specificity and/or affinity for said aberrantly expressed receptors.

Recombinant Patient Specific Tumor Receptor Ligand—a “best fit” protein having the highest relative specificity and/or affinity for the aberrantly expressed receptor

Specificity and/or Affinity is understood to mean when two tumor receptor specific ligands are contacted with a receptor, in vivo or in vitro, both having the same initial concentration, the one with the highest subtractive concentration after contact, is the one with the highest relative specificity and/or affinity.

Chemotherapeutic Agent: refers to cytotoxic antineoplastic agents, that is, chemical agents which preferentially kill neoplastic cells or disrupt the cell cycle of rapidly-proliferating cells, or which are found to eradicate stem cancer cells, and which are used therapeutically to prevent or reduce the growth of neoplastic cells. Chemotherapeutic agents are also sometimes referred to as antineoplastic or cytotoxic drugs or agents, and are well known in the art.

Chiral Index: Relative specificity and affinity of one ligand for a particular Gastrin Releasing Peptide Receptor (GRP-R) relative to another ligand wherein both ligands have the same number of amino acids but differ in sequences.

Gastrin Releasing Peptide Receptor (GRP-R) : A moiety having a heptahelical structure, wherein the Receptor-Ligand Attachment is focused in the third extra-cellular domain; said moiety having Active Amino Acids (aa): Phenylalanine (F), Serine (S) and Threonine (T), and wherein facing of aa's is inward within 5 Angstroms of the putative binding pocket. Interactions of the GRP-r are via hydrogen bonding and receptor-ligand cation-pie, and affinity is high with Kd of 1.5 molecules per nm³.

Receptor TRAP: A diagnostic kit that enables healthcare professionals to perform a presumptive screening test for a number of cancer forms to determine if additional testing and imaging procedures are needed.

SPECT Imaging: SPECT (Single Photon Emission Computed Tomography) is a nuclear medicine tomographic imaging technique using gamma rays. The technique results in a set of image slices through a patient, showing the distribution of a radiopharmaceutical. A patient is injected with a gamma-emitting radiopharmaceutical. Then a series of projection images are acquired using a gamma camera. The projection images are stored digitally and a sophisticated computer program is used to process them and produce the slices (this is called reconstruction). SPECT scans for oncology are supported by a number of radiopharmaceuticals that are specific to individual tumors. Tumor specific tracers include, gallium 67 for lymphoma, indium 111 octreotide for neuroendocrine tumors, thallium 201 for breast cancer and PROSTASCINT for prostate cancer. As opposed to PET (see below), SPECT does not have a non-specific imaging tracer, able to “capture” several tumor types in one scan.

PET Imaging: PET scan or positron emission tomography is a medical imaging technique that monitors metabolic, or biochemical, activity in the brain and other organs by tracking the movement and concentration of a radioactive tracer injected into the bloodstream.

The technique uses special computerized imaging equipment and rings of detectors surrounding the patient to record gamma radiation produced when positrons (positively charged particles) emitted by the tracer collide with electrons. PET scans are especially valuable in imaging the brain. They are used in medicine to diagnose brain tumors and strokes, and to locate the origins of epileptic activity; in psychiatry to examine brain function in schizophrenia, bipolar disorder, and other mental illnesses; and in neuropsychology to study such brain functions and capabilities as speech, reading, memory, and dreaming.

SPECT and PET differ from anatomically-based imaging modalities, such as MRI and X-rays, in that they assess the level of metabolic activity and perfusion in various organ systems. The process produces biologic images based on the detection of gamma rays that are emitted by a low dose radioactive substance such as the radioactive sugar FDG, which is the most common tracer used in conjunction with PET scans. The radioactive sugar can help in locating a tumor, because the faster growing cancer cells absorb sugar faster than other “normal” tissues in the body. FDG-PET has been shown to be effective in the staging of Hodgkin's and non-Hodgkin's lymphoma, and for staging of lung cancer and other tumors including cervical cancers. Improved disease staging can translate into better treatment decisions and overall prognosis.

Imaging Tracers: Imaging tracers refer to the radiopharmaceutical compound used in conjunction with SPECT and PET procedures to enhance the imaging process. Imaging tracers are commonly divided into three broad categories based on what they measure (1) tracers which provide general metabolic data, such as glucose uptake and protein synthesis, via labeled biomarkers (e.g., 11C-deoxyglucose and 11C-methionine, respectively) that leave the bloodstream and enter cells. These tracers are common in oncology applications. FDG, for instance, belongs to this category of tracers; (2) tracers which provide estimates for grosser physiological parameters, such as blood flow (e.g., 15O-H2O or 11CO2), and essentially remains in the bloodstream over the effective study duration, and are common in cardiovascular applications; and (3) tracers which delineate and quantify highly specific molecular targets, such as cellular receptors and transporters for which tracers are either endogenous ligands or drugs (e.g., 11C-raclopride for the DA2 dopamine receptor).

Patient Specific Ligands (PSL): ligands (proteins) that are specific to a cancerous cell and to at least one patient.

### BRIEF DESCRIPTION OF THE DRAWINGS

FIG. 1. This shows a schematic diagram of the steps used to classify GPR receptors in accord with the invention;

FIG. 2. This shows a schematic diagram of the steps of a method used to diagnose and treat patients in accord with the present invention;

FIG. 3 shows the amino acid (aa) sequence to produce a synthetic ligand that acts similar to the indigenous ligand found in the human body named gastrin releasing peptide (GRP) and further shows two groupings of aa's active in the affinity matching of this ligand to the corresponding GRP receptor in accord with the present invention;

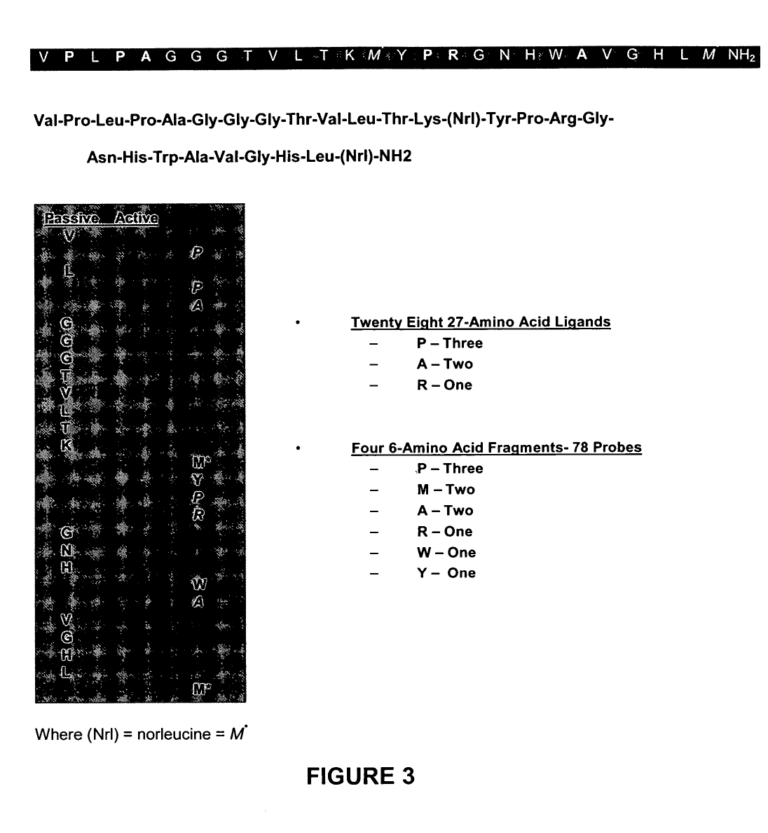

FIG. 4 shows three levels of ligand characterization of GRP-r in accord with the present invention;

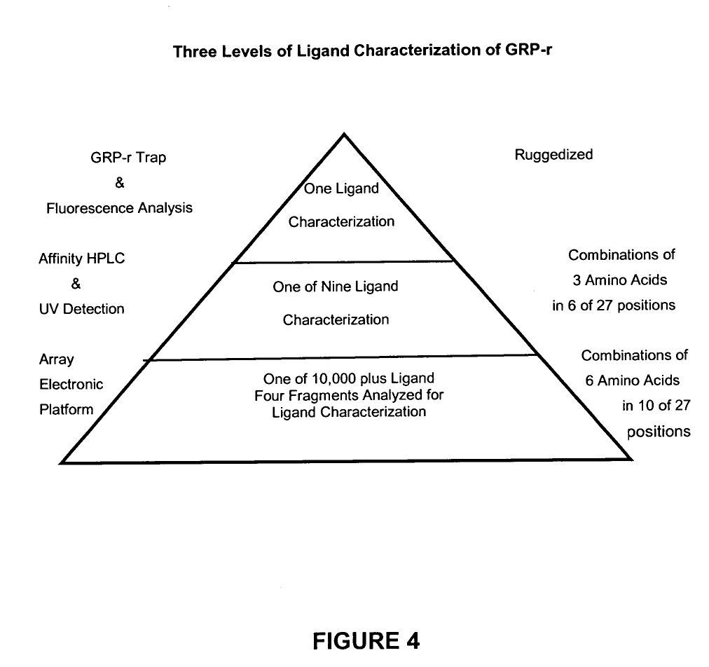

FIG. 5 shows a schematic diagram of steps used to determine the presence of GRP receptors in specimens of clinical fluids in accord with the present invention;

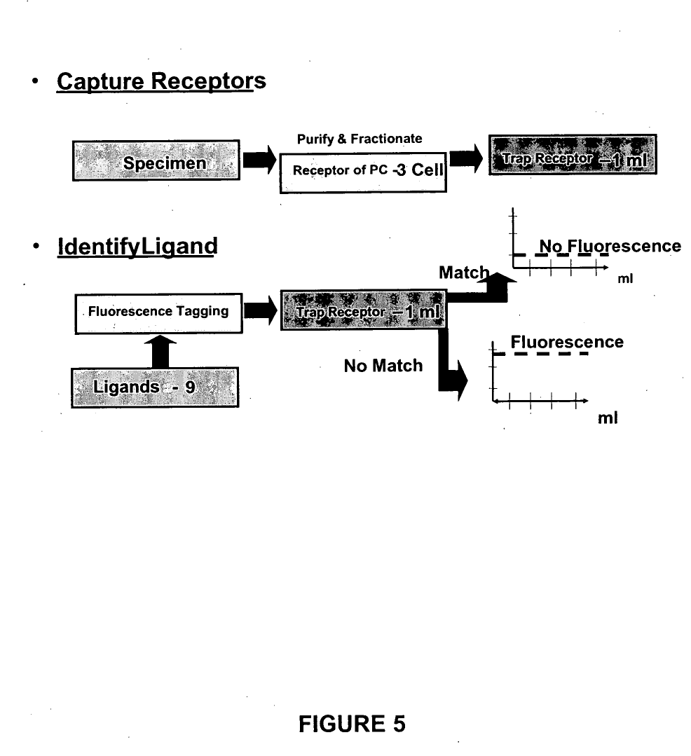

FIG. 6 shows the aa sequences for a population of twenty-seven ligands plus the ligand in Level One to produce a population of eight additional ligands chosen based on affinity matching to the corresponding GRP receptor to produce a total of nine ligands used to classify GRP receptors in accord with the present invention;

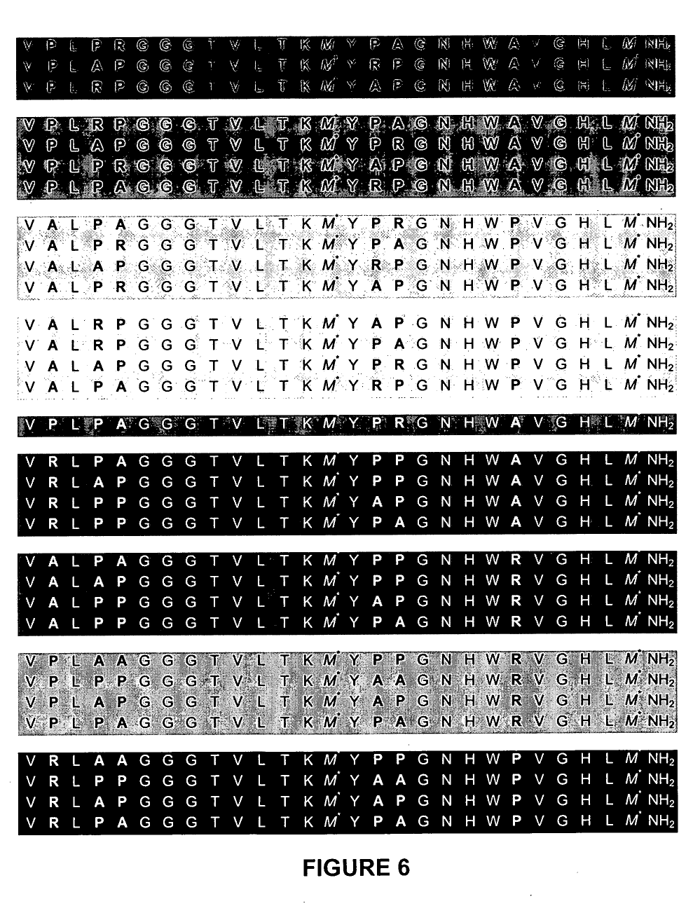

FIG. 7 shows a histogram of the frequency of occurrence of each of the nine ligands obtaining the maximum affinity matching to GRP receptors in patients with the ligand in FIG. 2 being the most frequent match in accord with the present invention;

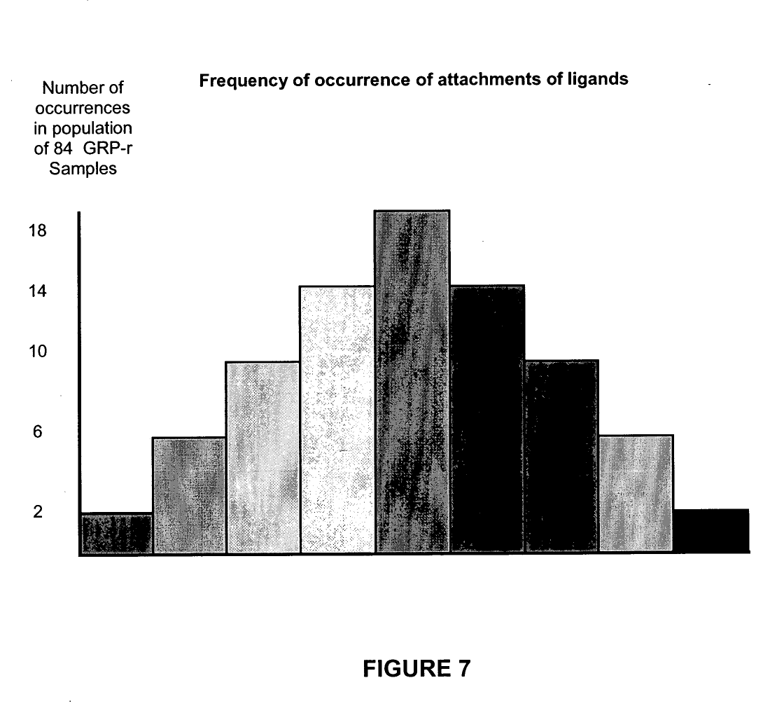

FIG. 8 shows a schematic diagram of the cross section of the cell used in electronic analysis platform in accord with the present invention.

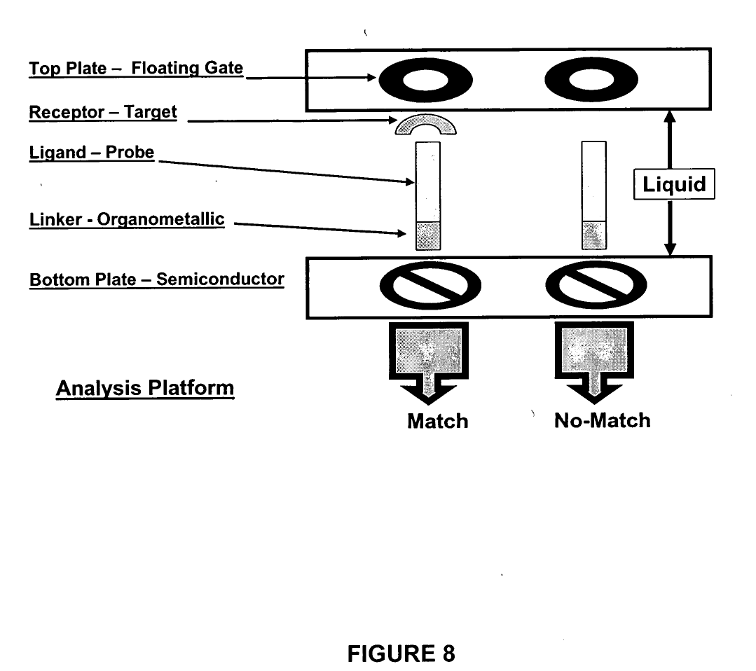

FIG. 9 shows a schematic diagram of the probes containing 78 fragments of the active aa in the 27 aa ligand in a 96 spot array and the contacting of said array of probes with the active fragment of the GRP receptor in accord with the present invention.

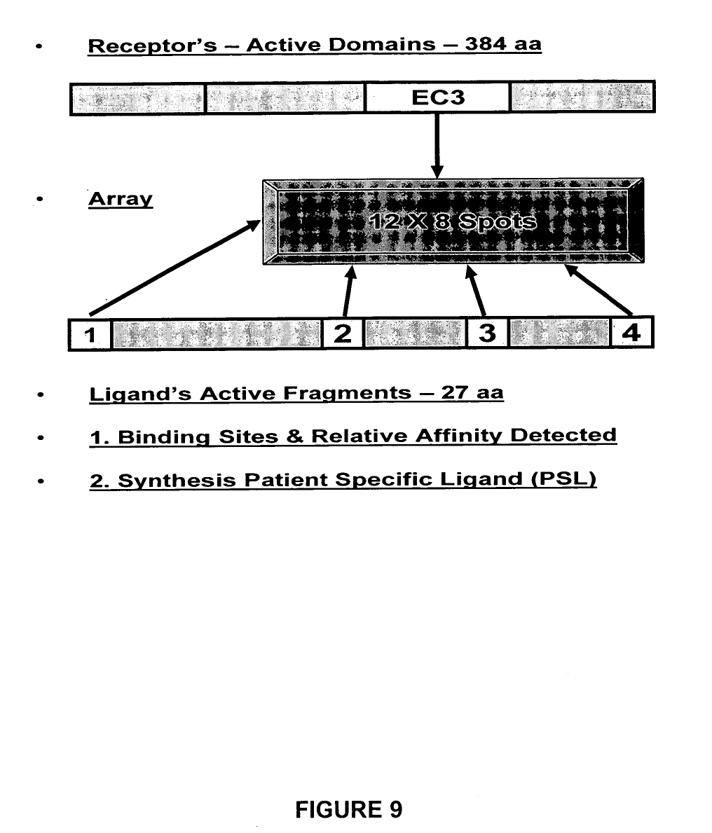

FIG. 10 shows the aa sequences for the population of 78 probes used to define the degree of affinity matching to a patient's specific GRP receptor by in vivo analysis so as to define a ligand with the maximum affinity matching by use of electronic signals from the probe points of the array contained in the electronic analysis platform in accord with the present invention.

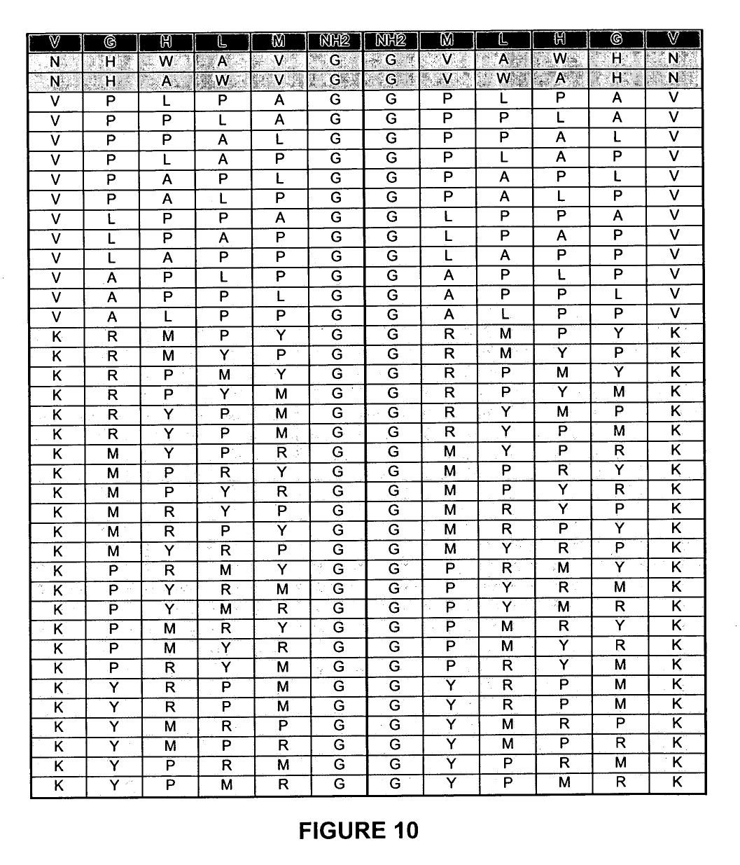

### DETAILED DESCRIPTION OF THE INVENTION

1. Overview

Utilizing a nanotechnology platform, the instant invention provides a unique method that enables identification and separation of ligands (proteins) that are specific to a cancerous cell and to each patient. For diagnostic applications, the ligands can, after separation, be replicated and tagged with either a radioactive agent for the SPECT and/or PET tracer or a fluorescence (dye) for the Receptor TRAP. The technology platform is exemplified as being based on gastrin releasing peptide receptors (GRP-R). Gastrin releasing peptides are being studied as biomarkers for a number of cancer forms including renal cancer, prostate cancer and lung cancer.

The invention is concerned with classifying tumor receptor subtypes as biomarkers and, for each biomarker, providing a library of ligands, which are generally agonists, that can be used to bind to the tumor in diagnostic and therapeutic capacities as further described herein. The invention is further concerned with providing a method whereby an individual patient's tumor can be diagnosed, after which personalized diagnostics and therapeutics can be designed using a pre-determined set of optimized ligand-based components. Section 2 below describes exemplary tumor receptors that form the basis for describing certain aspects of the invention. Section 3 below provides general methods for classifying tumor receptors, in accordance with one feature of the invention. Section 4 below describes the compilation and selection of ligands from ligand libraries. Section 5 below provides guidance for creating receptor-specific, ligand-based, diagnostic and therapeutic molecules. Section 6 below describes methods for diagnosing and treating individual patients.

2. Tumor Receptors

This section describes exemplary receptors expressed by tumors that may serve as biomarkers, targets, or both biomarkers and targets, in accordance with the present invention. Generally receptors that are suitable for use in the invention are membrane receptors characterized by the presence of at least one extracellular ligand binding domain. The specific receptors described in this section are exemplary and are not intended to limit the invention. Persons skilled in the art will recognize that other suitable receptors can be used according to the invention to target tumor cells.

Epidermal growth factor receptor (EGFR) is a 170-kDa cell membrane receptor consisting of an extracellular domain, a short transmembrane domain, and a cytoplasmic domain exhibiting a protein tyrosine kinase (PTK) activity. EGFR is also referred to as c-erb-B1. Binding of epidermal growth factor (EGF) to EGFR results in receptor dimerization with itself or with other members of the Erb-B transmembrane PTK family. Another member of this family, c-erb-B2, is also known as HER-2/neu, which is commonly associated with certain types of breast tumor cells and which is the target of Genentech's trastuzumab (HERCEPTIN®), a monoclonal antibody that binds the HER-2/neu receptor. Both EGRF and HER-2/neu are overexpressed in non-small-cell lung cancers, making them targets for therapeutics and/or diagnostics for this type of cancer, among other organ cancers.

Vascular endothelial growth factor receptor (VEGFR) is a membrane receptor that is normally present on the vascular endothelium. Like EGFR, VEGFR is a tyrosine kinase. Its ligand, vascular endothelial growth factor (VEGF), stimulates production of new blood vessels (angiogenesis)—and this includes the blood vessels that growing tumors need for their nourishment and growth. As a result, this receptor/ligand pair have been the focus of much interest by drug companies. For example, Genentech's bevacizumab (AVASTIN®) is a humanized monoclonal antibody to VEGF that is designed to bind and inhibit the binding of the ligand to VEGFR. It is currently being tested for use in renal cell carcinoma, advanced non-small-cell lung cancer, metastatic breast cancer, pancreatic cancer, and colorectal cancer.

More recently, it has become apparent that there is more than one form of VEGFR. These forms have been given designations VEGFR-1, -2, and -3; the designation of the form most commonly associated with angiogenesis is VEGFR-2. Knowing the presence and relative proportions of these subtypes may be important to both diagnosis and treatment.

The bombesin receptor family comprises at least four mammalian, G-protein associated, membrane receptor subtypes: gastrin receptor peptide receptor (GRP-R), neuromedin B receptor (NMBR), bombesin receptor subtype 3 (BRS-3), and bombesin receptor subtype 4 (BRS-4). Like most G-protein-associated receptors, each of these receptors is characterized by seven transmembrane domains, based on predicted amino acid sequence analysis. GRP-R has been shown to have a relatively high binding affinity for the 27-amino acid peptide known as gastrin releasing peptide (GRP); while NMBR has a relatively high affinity for the 25-25-amino acid, C-terminal, amidated peptide neuromedin (hence the first letter of its name).

In healthy humans, the highest expression of GRP-R is in the pancreas, where four different gene transcripts have been detected by Northern blot analysis. GRP-R is not normally expressed by epithelial cells lining the gastrointestinal (GI) tract, except for those lining the antrum of the stomach, where two gene transcripts have been detected. However, in contrast many GI tumors aberrantly express GRP-R, including as many as 40% of resected colon tumors (Carroll, R. E. et al., Mol. Pharm. 58: 601-607, 2000.) GRP-R is also expressed by certain other cancer cells, such as small-cell lung cancer cells, and by prostate tumors (Ferris, H. A. et al., J. Clin. Invest. 100: 2530-2537, 1997). GRP-R have also been detected in certain tumor cells in culture (e.g. breast, lung, and duodenal cancer cells.)

Binding of GRP to GRP-R can cause the proliferation of many, but not all, cells in which the receptor is expressed. Thus, GRP acts as a mitogen for certain tumor cells, such as adenocarcinoma cell lines of breast and prostate origin, as well as in small-cell lung cancer cells. While under normal circumstances GRP-R activation is dependent upon binding of GRP (or another GRP-R agonist) to the receptor, certain mutant forms of GRP-R have been found that are not dependent upon ligand binding for activation. Such receptors are constitutively activated, as evidenced by GRP-independent growth cycles. (Ferris, H. A. et al., supra.)

Additional tumor receptor biomarkers suitable for use in the present invention can be identified by the practitioner, with reference to one or more available review articles in the field (e.g. Tsao, A. S. et al., C A Cancer J. Clin. 54: 150-180, 2004).

3. Classification of Tumor Receptors.

According to an important feature of the present invention, tumor cell receptors, such as GRP-R, are classified by best fit ligand, to provide a tumor-specific receptor subtype profile. According to a further feature of the invention, common receptor mutants or subtypes can be isolated and classified to provide a GRP reference tumor receptor library. Patient tumor samples are compared to the library, and classified according to their receptor similarities to reference tumor receptor subtypes, to provide a tumor GRP-R profile. This section will provide guidance for various ways of classifying tumor receptors to generate such profiles.

A. Column Chromatography

Membrane-bound receptors are generally tightly associated with membrane lipids. Successful isolation and purification of GRP-R usually depends upon selection of an appropriate detergent solubilizing agent, which must be compatible with receptor binding activity as well as with the various further purification steps, such as column chromatographic matrices to which the receptor protein will be subjected. Specific conditions and methods for isolation and purification of certain membrane receptors are known in the art, as exemplified below. Such receptors can be purified and used as standards for comparative purposes. Alternatively, receptors having known coding sequences can be made artificially and expressed in cells that are capable of post-translational modifications (glycosylation), such as SF9 insect cells (Kusui, T. et al., Biochemistry 34(25): 8061-8075, 1994) and purified according to methods known in the art.

The membrane receptor GRP-R has been solubilized from various cell types. For example, the receptor was solubilized from human small-cell lung carcinoma NCI-H345 cells using 0.1% Triton X-100 or dodecyl-□-D-glucopyranoside (0.05%) as solubilizing detergent (Kane, M. A. et al., J. Biol. Chem. 266: 9486-9493, 1991) and from human glioblastoma (U118) and lung carcinoid (NCI-H720) cell lines using CHAPS/cholesterol hemisuccinate as solubilizing agents (Staley, J. et al., Neurosci. 4(1): 29-40, 1993). Further purification can then be achieved using an affinity ligand column. By way of example, Staley et al., supra, were able to achieve 85,000-fold purification of the GRP-R from human tumor cells by using (Lys0, Gly1-4, D-ala5)Bombesin and (Lys3, Gly4, D-Tyr6)Bombesin 3-13 propylamide affinity resins in tandem.

Similar solubilization and purification of the receptor have been achieved using a tissue source (rat pancreatic particulate membranes; Kane M. A. et al., Peptides 12(2): 207-213, 1991), demonstrating the applicability of such techniques to patient samples, as contemplated by the instant invention. Additional affinity resins may be formed by methods well known in the art, using ligands selected as described in Section 4 below, in order to purify tumor-specific receptor subtypes in accordance with the present invention.

In one embodiment, the invention contemplates a “grid” approach to affinity selection, whereby receptor material is subjected to affinity chromatography on “pooled” affinity ligand columns, in which pluralities of ligands are mixed in affinity matrices to maximize the potential for binding. Following elution of bound receptor, the identity of the binding ligands can be determined by successive rounds of re-chromatography on matrices having fewer ligand members.

Other forms of column chromatography are available as classification modes. For example, the ligand-affinity purified receptor can be subjected to affinity chromatography. Selection and optimization of such techniques can be achieved empirically without undue experimentation on the part of the skilled artisan.

Generally, the relative elution pattern of receptor material is monitored and recorded, in order to characterize a particular receptor subtype. Monitoring of eluted material can be achieved by one or more methods well known in the art, including but not limited to I-ligand binding to elution fractions, silver-stain gel analysis, western-blot gel analysis, and their like.

B. Ligand-binding Properties

Specificity of ligand binding can be used to distinguish among various GRP-R subtypes, particularly those that are characterized by mutations within the third extracellular domain (Tokita, K. et al., Mol. Pharm. 61: 1435-1443, 2002). By way of example, assays for GRP-R ligand binding specificity, such as the use of competitive binding assays to determine rank order of ligand binding specificity among ligands, are well known in the art. For example, GRP-R binding selectivity, using radiolabeled bombesin as radioligand, is preferential (higher affinity) for GRP over bombesin; and both radiolabeled bombesin and bombesin are similarly preferential over neuromedin (GRP >bombesin> neuromedin). Refinement of such techniques may be achieved by selecting additional peptide ligands according to the methods set forth in this specification and the incorporated references. Other methods of determining ligand specificity include techniques in which specific peptide ligands are attached vial linker molecules to substrates, such as wells or pins, suitable for mass screening. Custom-designed, immobilized peptide libraries are commercially available. For example a custom peptide library designed to “analog” the sequence of GRP as a custom Pepset™ can be obtained from Mimotopes (San Diego, Calif.) on polyethylene pins mounted on blocks in a format that is compatible with standard 8×12 microtiter plates. Such peptides are incubated with receptor mixtures, and bound receptor measured, for example by enzyme-linked immunosorbent assay (ELISA) techniques known in the art.

For smaller scale ligand specificity determinations, a nanoarray format peptide library may be preferred, consisting of 96 wells, each 0.1 mm diameter in an 8×12 format. Defined peptides are found to each well via a linker molecule to form an addressable molecular “ligand probe”. The target receptor (e. g. GRP-R) is flooded onto an array at a concentration of approx. 200 pg receptor per square millimeter of surface area. In a nanoarray having an active area of 117 mm2 (e.g. a 9 mm×13 mm square plane), approximately 23.4 nanograms receptor (approx. 540 fmoles GRP-R) is used to flood the surface of the array. Then the flooded array is incubated. Following incubation, excess receptor is washed away. Binding to the ligand probes can then be measured, for example by a change in capacitance between the ligand probes, submerged in an ionic solution, such as 0.15M NaCl, used to wash away the excess receptor.

Binding of GRP-R to a ligand probe results in a difference in capacitance compared to the norm (which is defined as the capacitance between the pre-flooding, unbound ligand probes). The location in the array of the ligand probes, can be determined by triangulating the position of the ligand probe based on capacitance measurements from adjacently located probes. The two ligand probes and GRP-R attach in a dipole interaction called a “salt bridge”. The energy Ee is given by Coulomb's law as:
Ee=((e²/4 Pi)* 8.84*10⁻¹²) (Z₁* Z₂/kₑ*r) exp (−K_{dh}*r);
Where e=1.16*10⁻¹⁹ coulombs; Z₁ and Z₂ are the numbers of attractive charges, r is the distance between the charges, and kₑ is the dielectric constant. For (human) blood plasma or interstitial fluid, which is approximated by 0.15M NaCl, where the two charges are separated by 0.3nm, the interaction energy Eₑ=6.3 zJ.

In an alternative embodiment, the location in the array can be determined by a matrix differentiation in electrical characteristics for each node through a backplane test of the array of wells,, where every well is wired and each well forms a node, that treats each well as a potential capacitor and identifies those which, due to ligand binding, are differentiable from the non-binding wells, similar to or even using means used to test semiconductor EEPROM or PAL circuitry.

Ligand binding specificity can also be determined using peptide phage display library technology. Briefly, the filamentous bacteriophage virion consists of a single stranded DNA strand surrounded by a major coat protein pVIII. Up to five copies of a minor coat protein, pIII, are present at the tip of the virion. Vectors encoding pIlI can be engineered to display “foreign” amino acids (peptides) according to methods well known in the art (e.g. Cwirla, et al., Proc. Natl. Acad. Sci. 87: 6378-6382, 1990). A “library” of 20ⁿ possible sequences (where “n” represents the number of amino acids in the peptide) can be generated. Random libraries of linear 12-mers and linear or cyclized 7-mers are commercially available from New England Biolabs (Beverly, Mass.); methods for producing longer peptide inserts, including cyclized (cysteine constrained) and positional amino acid constrained peptides are also known in the art (see Wang, B. et al., Biochem. 38: 12499-14509, 1999; Rainey, M.A. et al., J. Phys. Org. Chem. 17: 461-471, 2004; Bach, M. et al., Prot. Eng. 16: 1107-1113, 2003).

Binding preference for a specific peptide sequence may be carried out using a “peptide panning” technique known in the art. Briefly, the receptor target molecule (e.g. GRP-R) is coated onto an immobile surface, such as a bead or a plate. A mixture of phage-bearing random peptides is incubated in contact with the receptor-coated surface, then the surface is washed to remove unbound phage. Bound phage are then eluted away from the surface, according to methods known in the art, and used to infect host bacterial cells, where the phage are then amplified. The amplified phage are panned again by re-incubation with the target, and the process is repeated 3-4 times to successively enrich the pool of phage in favor of the tightest binding sequences. Phage clones are then isolated, and individual clones are characterized by DNA sequencing to determine the DNA sequence of the insert and, thereby, the amino acid sequence of the selected peptide(s). Peptides can be synthesized by methods known in the art (such as F-moc based solid-phase synthesis) and purified by high-performance liquid chromatography (HPLC) for further characterization. Assuming that more than one peptide is identified by such a procedure, the characteristic of the mixture may be used as an identifier; alternatively, the component peptides can be further tested and differentiated for rank-order of binding, to classify the receptor subtype.

4. Receptor-Specific Ligands and Libraries

This section will provide guidance for creating and identifying receptor-specific and/or receptor-optimized ligands for use in the invention. Such ligands can be used to further characterize, identify, and/or classify subtypes of tumor receptors; and may further be used as components for diagnostics and/or therapeutics, as described in Section 5, below.

Many membrane receptors have as their endogenous ligands relatively short peptides (generally no greater than 50-75 amino acids in length). Such peptides may fold into three dimensional structures that conform to the active binding site of the target receptor. In cases where mutations to a receptor affect its ligand binding site, novel ligands may bind where endogenous ligands lack requisite binding affinity. In accordance with one aspect of the invention, libraries of peptide ligands can be screened for candidate peptides that bind to the receptor with enhanced affinity, or bind to deficient and/or altered, mutant, receptors, or bind preferentially to altered or mutant receptors. These novel ligands may include synthetic ligands.

In accordance with one aspect of the invention, libraries of peptide ligands can be screened for candidate peptides that bind to a specific receptor subtype with enhanced affinity, bind to deficient and/or altered, mutant, receptors, or bind preferentially to altered or mutant receptors. Such peptide libraries can be conveniently generated by a known methodology known as “phage display” as described in Section 3.B supra, or can be purchased from commercial sources, as described below.

Generation of peptides by the phage display method has the advantage that billions of peptides can be screened for affinity to a receptor subtype. Briefly, the same panning method described in Section 3.B is applied—receptors bound to a solid phase are contacted with mixed phage, then non-binding phage is washed away. Phage that bind to the receptor are eluted and amplified by re-inoculation of the host bacterial cells. The pIII insert DNA is sequenced to determine the deduced amino acid sequence of the peptide insert(s) of the phage that bind the receptor. Corresponding binding peptides are then synthesized using common synthetic methods as described above. Binding properties of these ligands are then further characterized, individually or as batches, as described below.

Alternatively, peptide mixtures are commercially available as peptide screening libraries (e.g. PEPscreen™ from Sigma/Genosys, St. Louis, Mo.; PepSets™ from Mimotope, San Diego, Calif.). Generally, such libraries will be formed using as a starting point some portion or portions of the endogenous ligand, such as GRP. These mixtures can be provided on solid phases, for screening as described in Section 3.B supra. Alternatively, they can be provided as mixtures of peptides, which are then screened using an HPLC classification method.

Briefly, the mixture of peptides is separated by HPLC to produce classification windows corresponding to effluent fractions. Such fractions are then tested for binding to receptor subtypes to produce a one-to-one correspondence to the receptor subtypes.

In the event that small molecule ligands are desirable, libraries of organic molecules can be screened for competition of binding of a known ligand, such as a peptide ligand, as described above, using a standard competitive binding assay system, according to methods known in the art. Alternatively or in addition a method can be practiced in which the phage peptide display library is modified to permit attachment of synthetic organic compounds to the phage coat proteins. This method allows for later deconvolution and identification of the synthetic compound(s) of interest (Woiwode, T. F. et al., Chem. Biol. 10: 847-858, 2003).

5. Receptor-Specific, Ligand-Based, Diagnostic and Therapeutic Compositions And Methods.

This section describes the use of the ligands described above to produce diagnostic and therapeutic compositions, in accordance with the invention.

In one embodiment, it is an objective of this invention to teach a process for producing a recombinant patient specific tumor receptor ligand, and the product produced thereby, according to the following steps:
providing a serum or tissue sample obtained from a host and suspected of containing cancerous cells;
providing a plurality of species of ligands to plural types of receptors, which ligands species are indigenous to said host, and wherein each of said species is known to bind to a receptor found on a cancer cell;
confirming binding of at least one of said species of host indigenous ligands to a cancer cell;
providing a plurality of tumor receptor specific ligands associated with said bound indigenous ligand, wherein said tumor receptor specific ligands exhibit varying degrees of specificity and/or affinity for aberrantly expressed species of said receptor present on said cancer cell;
deriving a subset of patient specific tumor receptor ligands from said tumor receptor specific ligands; and
deriving or determining a recombinant patient specific tumor receptor ligand;
wherein said recombinant ligand is characterized as exhibiting the highest relative specificity and/or affinity with respect to said subset of patient specific tumor receptor ligands; and
whereby providing a ligand equal to said recombinant patient specific tumor receptor ligand is useful in diagnostic and therapeutic applications associated with cancer.

Within the context of the above outlined procedure, the terms utilized have the following meaning:

Ligand: A molecule that binds to another. Often, a soluble molecule such as a hormone or neurotransmitter that binds to a receptor.

Indigenous Ligand—a naturally occurring protein, which may be an agonist or an antagonist, and which binds to a particular receptor type in mammals.

Tumor Receptor Specific Ligands—a plurality of proteins associated with and including said indigenous ligand and characterized as having different sequences but the same number of amino acids as the indigenous ligand, and which bind to aberrantly expressed receptors on cancerous cells with varying degrees of specificity and/or affinity.

Patient Specific Tumor Receptor Ligands—a select group of Tumor Receptor Specific Ligands exceeding a minimum specificity and/or affinity for said aberrantly expressed receptors.

Recombinant Patient Specific Tumor Receptor Ligand—a “best fit” protein having the highest relative specificity and/or affinity for the aberrantly expressed receptor

Specificity and/or Affinity is understood to mean when two tumor receptor specific ligands are contacted with a receptor, in vivo or in vitro, both having the same initial concentration, the one with the highest subtractive concentration after contact, is the one with the highest relative specificity and/or affinity.

A. Diagnostics

Classification of tumor receptor biomarker subtypes, as described in Sections 3 and 4, provides a basis for rapid, accurate, and patient-and-tumor-specific diagnosis of receptor subtypes present on any particular patient tumor(s) and in patient serum samples, as described below. Material from a particular patient sample (from a biopsy, serum sample, or both) is subjected to at least one of the classification steps as described in Section 3 above, selected to provide a profile of the tumor receptor of interest. This profile is compared to an archive or library of biomarker sub-type information to determine a definitive or at least most-strongly present and probable tumor receptor subtype in the patient sample. Identification of tumor receptor subtype facilitates diagnosis and subsequent treatment, based on historical performance of treatment regimens against tumors exhibiting the same or similar receptor subtype.

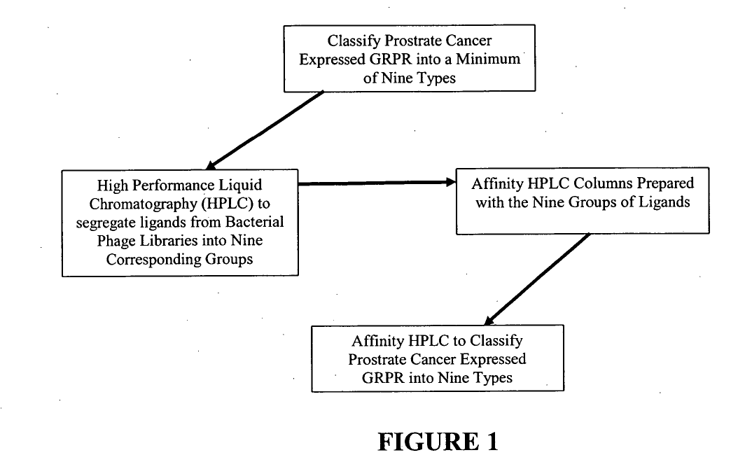

FIG. 1 shows a schematic flow diagram of an exemplary analysis in accordance with the present invention. In this diagram, as one example, prostate-specific GRP-R are classified on the basis of Affinity HPLC chromatography, are separated into at least nine pooled groups, and affinity columns are prepared using these pooled ligands. A patient sample is then prepared and subjected to the chromatographic steps described above. Its binding profile is compared to previously characterized tumor receptors to provide an accurate, definitive diagnosis of the GRP-R subtype.

A further embodiment of the invention provides additional diagnostic compositions, based on the foregoing technology. Alternatively or in addition to the paradigm described in the previous two paragraphs, filamentous phage-bearing peptide ligands identified and pooled as described above can be used to directly bind to tumor biopsy samples in a phage overlay assay format (Zurita, A. J., et al., Canc. Res. 64: 435-439, 2004).

Receptor subtypes present in patient serum samples may also be classified, according to the principles discussed above; however, since it is contemplated that one or more orders of magnitude greater sensitivity will be required for analysis of serum samples (due to their relative dilution as compared to biopsy samples), detection of receptor subtypes in serum samples is preferable carried out using a nanoarray detection methodology, such as those detailed in Section 3.B above. Briefly, a peptide library is formed in the wells of a nanoarray, each well containing a unique peptide bound to the substrate via a linker molecule to form an individually-addressable, molecular, ‘ligand probe’ in an array of differentiated ligand probes. The serum sample may be concentrated prior to adding it to the array, to achieve a detectable concentration receptor (approx. 100-200 pg receptor per square millimeter of array surface). Following incubation, excess receptor is washed away. Binding to the particular ligand probe(s) is measured, by differential capacitance between the ligand probes, as discussed in detail in Section 3.B.

In a further embodiment of this invention, cross-comparison between more than one diagnostic may be used to narrow further the patient-specific ligand(s) and/or provide relative proportions for multiple ligands or differentiated samples.

In yet a further embodiment of this invention, a ligand may be compounded with a material reactive to a particular diagnostic or therapeutic input, with such input potentially coming from outside stimulation of the ligand or compounded material.

In still yet a further embodiment of this invention, the receptor-specific, ligand-based, diagnostic and therapeutic compositions and methods may use only a part of a ligand, that part being as much as is needed for the required specificity.

In still yet a further embodiment of this invention, the receptor-specific, ligand-based, diagnostic and therapeutic compositions and methods may use all or parts of more than one ligand, as needed for the required specificity.

B. Therapeutic Compositions

Another specific feature of the present invention provides therapeutic compositions designed to target the specific GRP-R subtypes identified as described in the preceding sections. Generally, therapeutic compositions in accordance with this feature of the invention are formed from a GRP-R-subtype-specific ligand that is conjugated to a tumor ablative compound, such as a known chemotherapeutic agent or radioactive moiety.

In a specific embodiment, the therapeutic composition is formed from a filamentous bacteriophage, such as fd-tet based bacteriophage vector, bearing a GRP-R-subtype-specific peptide ligand inserted into the pIII coat peptide, as described above. The chemotherapeutic agent may be inserted into the internal “core” formed by the bacteriophage core filament (protein pVIII); alternatively, the chemotherapeutic agent may be conjugated to the bacteriophage itself, using the methods described by Woiwode, T. F., et al., Chem. Biol. 10: 847-858, 2003. Other methods of conjugating GRP-R-subtype-specific ligands to chemotherapeutic agents will be readily ascertainable to those skilled in the art, based on the chemical composition and chemical reactivity of the agents to be conjugated.

As used herein, the term chemotherapeutic agent refers to cytotoxic antineoplastic agents, that is, chemical agents which preferentially kill neoplastic cells or disrupt the cell cycle of rapidly-proliferating cells, or which are found to eradicate stem cancer cells, and which are used therapeutically to prevent or reduce the growth of neoplastic cells. Chemotherapeutic agents are also sometimes referred to as antineoplastic or cytotoxic drugs or agents, and are well known in the art.

Exemplary chemotherapeutic agents include, but are not limited to, alkylating agents (cyclophosphamide, mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and gramicidine D), paclitaxel, colchicine, cytochalasin B, emetine, maytansine, and amsacrine. Additional agents (some of which, e.g. busulfan, may also fall into one or more of the categories listed above) include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate.

Other suitable agents are those that are approved for human use, including those that will be approved, as chemotherapeutics or radiotherapeutics, and known in the art. Such agents can be referenced through any of a number of standard physicians' and oncologists' references (e.g. Goodman & Gilman's The Pharmacological Basis of Therapeutics, Ninth Edition, McGraw-Hill, N.Y., 1995) or through the National Cancer Institute website (http://www.fda.gov/cder/cancer/druglistfrarne.htm), both as updated from time to time.

While not wishing to ascribe to a particular theory, it is believed that a phage-based, conjugated chemotherapeutic formed as described herein may be taken up by target tumor cells, by receptor internalization or endocytosis. Whether or not cellular uptake occurs, the specific targeting achieved by compositions of the present invention is expected to result in reduced overall drug toxicity, due to the reduced concentrations required to achieve a critical cytotoxic concentration of tumor ablative agent in or around the target tumor cell, and also the greatly (and relatively) reduced concentrations around non-receptive, non-tumor cells in the patient's tissues, even for the same organ and especially for non-targeted cell types. As such, therapeutics of the present invention may include potent compounds that have fallen into disuse, are disfavored, or have not been approved to use, based on their undesirable side effects and toxicities at the (non-specific targeting's) higher dosage concentrations.

Radiotherapeutics and other direct-contact metallic elements serving as tumor ablatives may also be used in the present invention. Generally, radionuclides suitable for use in peptide conjugates in accordance with the present invention will include those having suitable emission properties to provide tumor ablation in situ, while not unduly exposing the surrounding, non-cancerous tissues to damaging levels of irradiation. Thus, a radioactive tumor ablative compound can be made by conjugating a patient-specific ligand (PSL) to a radionuclide. According to general principles, an ideal radionuclide for use in such therapeutic compositions is a relatively short-lived alpha emitter, a gamma emitter, or a beta-emitter that emits enough gamma irradiation to cause local destruction.

Examples of radioisotopes that form this latter type of radionuclide include lutetium-177, iodine-131, iodine-125, and phosphorus-32. Alpha-emitters that have been used in cancer therapy, and are therefore suitable for use in the radioisotopes, include actinium-225, astative-211, and bismuth-212 and bismuth-213. Other useful radioisotopes, which have been used in cancer therapeutics, include iodine- 123, copper-64, iridium-192, osmium-194, rhodium-105, samarium-153, and yttrium-88, yttrium-90, and yttrium-91.

According to a related technique, boron-10 can be concentrated in tumors as part of a composition of the present invention. The patient can then be subjected to neutron irradiation, during which the neutrons are far more strongly absorbed by the boron than the organic tissues, causing the boron to produce intensely localized, high-energy, tumor-ablative alpha particles in situ.

The foregoing therapeutic compositions of the invention can be administered in any of a variety of pharmaceutically acceptable excipients known in the art. While such compositions may be administered by any of a number of modalities (including without limitation intravenous, intra-arterial, oral, intranasal, nasal insufflation, intramuscular, intraperitoneal, intrathecal, intraspinal, intracerebrovascular, or the like), direct vascular administration (intravenous or intra-arterial) or surgical implantation/injection are generally considered to be the most likely routes of administration. Accordingly, it is contemplated that compositions of the invention would be administered in a normal saline- or buffered-saline excipient.

Determination of the precise dosage of therapeutic composition can be made with reference to general pharmaceutical principles, taking into consideration that the targeted agent should concentrate at tissues bearing the targeted receptor(s). Hence, appropriate initial dosages can be determined with reference to experimentally determined first-pass effects from appropriate model systems. Generally, however, it is contemplated that dosages in the range of 0.001-10,000 mg/kg body weight will achieve the therapeutic objectives; narrow ranges may also be defined; 0.001-10 mg/kg; 0.01-100 mg/kg; 1.0-1000 mg/kg; 10-10,000 mg/kg; 0.1-10,000 mg/kg. In general, it is contemplated that the effective dosages will be no more than a ten-thousandth, a thousandth, or no more than a tenth of current, standard chemotherapeutic dosages of the conjugated chemotherapeutic agent, due to the targeted specificity of the binding therapeutic compositions of the present invention.

By way of example, vinblastine sulfate is a well-known drug, which can be an effective treatment for recurrent testicular cancer. When administered intravenously at a single dosage of 0.3 mg/kg of body weight, vinblastine sulfate causes myelosuppression and concomittant leucopenia, which can limit its dosage, along with other serious and toxic side effects including neurological effects (numbness of extremities, loss of deep tendon reflexes, muscle weakness), that discourage, even deter, or prevent some patients from receiving the treatment. In therapeutic formulations of the present invention, it is contemplated that much lower dosages (such as 0.0003-0.03 mg/kg body weight), a tenth to a thousandth less debilitating, would be effective.

By way of further example, mitoxantrone can be used to treat hormone-refractory prostate cancer. While this drug apparent exhibits less cardiac toxicity than its parent analog, doxirubicin, its dosing schedule is limited by acute myelosuppression and mucositis, and it is indicated for use at a dosage of 12 mg/m² body area in combination with a steroid (prednisone). In accordance with the present invention, it is contemplated that the dosage would be from 0.012-1.2 mg/m² body area, again a tenth to a thousandth as toxic to the patient (as opposed to the tumor).

6. Diagnosis and Treatment of Patients

According to an important feature of the present invention, patients will receive individualized diagnosis and treatment, in a manner that shifts the control and expenditure for cancer care away from pharmaceutical companies. Instead, care will rely on a decentralized integration of care management between the local clinician (and/or oncological specialist), who will take on responsibility not only for diagnosing the patient at the molecular level, by identifying the patient's tumor-specific receptor subtype, but also for selecting a patient-specific-ligand (PSL) best suited for that tumor-specific receptor subtype, then compounding the appropriate PSL-conjugated diagnostic and therapeutic agents. This localization, democratization and decentralization of pharmaceutical research and development is contemplated to tremendously reduce overall costs and greatly speed up progress along the learning curve for cancer care.

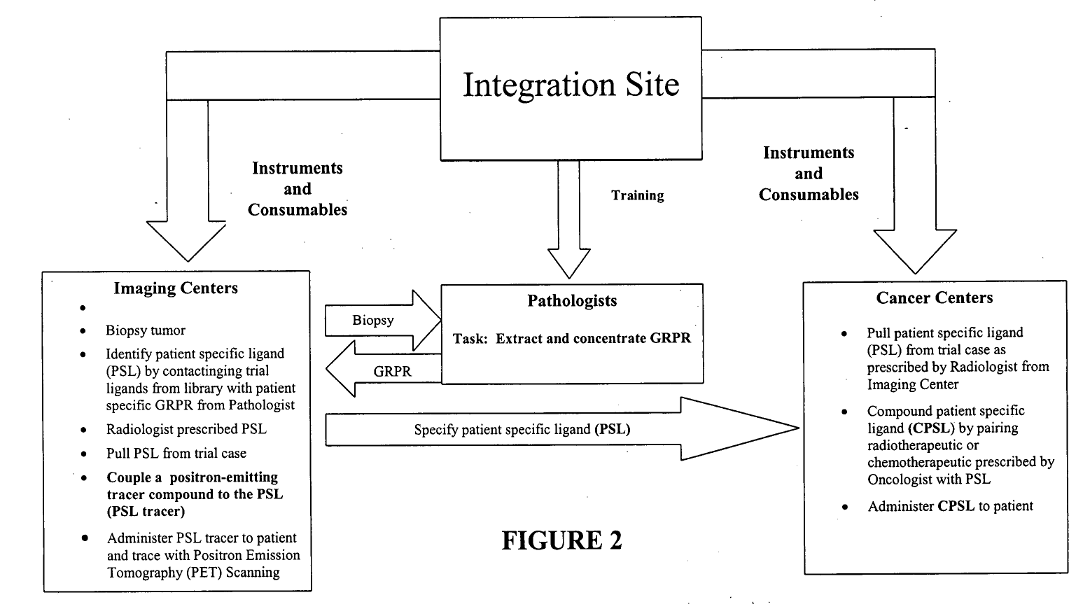

FIG. 2 shows a schematic diagram of a method for diagnosing and treating cancer patients in accordance with the present invention. A patient suspected of having a tumor is directed by his or her physician to an imaging center, where a biopsy or serum sample of the suspect tissue is taken, quite possibly, through arthroscopic or other minimally-invasive means, as a low-risk outpatient procedure. The sample is then subjected to receptor identification and classification, according to the method set forth above. Based on the receptor and receptor-subtype classification, a tumor-specific receptor ligand is chosen from a library of ligands. This step may optionally include step-wise exposure of the sample's receptors to successively more accurate and narrower pools of ligands, as described above.

Once an appropriate ligand has been selected for the patient's tumor—which has had the receptor and receptor-subtype classified—this patient-specific-ligand (PSL) is conjugated to an imaging agent, such as a positron-emitting radioligand suitable for positron-emission-tomography (PET) scanning or SPECT, thereby creating a PSL-PET-tracer. The PSL-PET-tracer is then used in the patient, initially to provide the pre-treatment baseline assessment of the degree and rate of tumor distribution (swiftly identifying any metasticized tumors or incipient tumors), though it may also provide further validation of the choice of PSL. This initial visualization may optionally then be used to fine-tune the initial therapeutic option (choice of agent and dosage regimen). Generally, it is appreciated that an effective imaging agent requires at most the same amount as a first treatment amount, but often may require no more than a half to a fifth that amount—this step also provides information as to the effectiveness of the targeting and penetration of the PSL agent.

Suitable isotopes for use in PET scanning are known in the art. Examples of such isotopes include but are not limited to technetium-99, arsenic-72, bromine-75, carbon-11, cobalt-55, cesium-137, copper-61, copper-64, fluorine-18, germanium-68, manganese-52, lead-203, rubidium-97, rubidium-103, and scandium-46. A plurality of tags for different ligands with maximum affinity for different cancer cell expressed GRP.

In parallel, the selected PSL is compounded into a therapeutic composition, linking the PSL to a tumor-ablative agent (chemical or radiological or biological) such as the phage-based therapeutic composition described in Section 5 above.

According to an important feature of the invention, such compounding is carried out as locally as possible and is specific to the individual patient, using standardized methods and reagents, rather than depending on ‘big-pharma’ broad-scale, non-specific compositions. The PSL therapeutic composition is then administered according to the precision-guided treatment dosages and regimen determined by the local clinician. The treatment regimen is then monitored by the local clinician with the steps (classification, PSL composition creation, scanning, dosing) being repeated if and as necessary.

In a further embodiment of this invention, the steps of classification, PSL-formation, therapeutic composition formulation, and treatment, are interwoven in accordance with the balance between the severity of the need for immediacy and the then-current accuracy of the diagnosis. As the diagnostic compositions and processes provide increasingly accurate targeting, the clinician provides more specifically targeted (and thus more effective, yet less deleterious) treatment specific to the patient, tumor, tumor receptor, tumor-receptor-subtype(s), and tumor-receptor-subtype-therapeutics. Instead of operating with a ‘fire and forget’ therapeutic administration of each dose, the clinician's role becomes more closely analogous to an operator of a wire-guided missile, ever more closely homing in on the intended target—with far lower ‘collateral damage’. Feedback is continually used, not just during the diagnostic steps, but from all steps, to effect the best and least disruptive treatment.

In a further embodiment of this invention, the diagnosis, preparation, and treatment considers the relative value of negative matches against normal healthy tissues as one of the weighting factors to guide the PSL-linkages, seeking to maximize the true positives and minimize any ‘false positives’ that could misdirect the effect to surrounding healthy cells with like receptor subtypes.

Synthetic Ligand Characterization and Tagging Extra-cellular Receptors

The ability of a GRP receptor to bind to a plurality of ligands, all with the same linear chain length and number of amino acids but each ligand Chirally unique, resulting in measurable differences in the degree of specificity and matching affinity that is used to first classify a patient's GRP receptor, then synthetically produce the matching ligand with some or ideally the maximum Chirally and then determine the presence of the GRP-r by chemical-optical or radiopharmaceutical tagging said GRP receptor with said ligand.

According to a feature of the invention the patient's GRP-r in extracted from clinical fluids consisting of tissue, plasma or urine. The steps for extraction of the patient's GRP-r from tissue comprise: composite tissue sample, separation of GRP-r from tissue: enrichment of GRP-r: re-suspension, mining the active fragment of the GRP-r.

According to another feature of the,invention the extract from the patient's GRP-r is contacted with synthetic ligands or segments of synthetic ligands in three levels with each successive level defining increased specificity by deterring the match between synthetic ligands and an extraction of the patient's GRP receptor. The Level One ligand is a ruggedized by substituting the synthetic compound Norleucine for Methionine in the 27 mer ligand indigenous to normal human pancreas. Additional ruggedization of the ligand in the future is possible by finding a synthetic substitute for the amino acid Threonine to increase the shelf-life in aqueous solution from the present limit of three days. The Level One ligands is linear, has 27 positions for amino acids and contains thirteen amino acid types out of a total population of twenty possible amino acids. The Level Two population of possible ligands consists of 27 plus the Level One ligand and said population is defined using combinations of the three active amino acids with first-order Chiral activity (Proline, Alanine and Arginine) that occur in six of the 27 positions. This population is reduced to a total population of nine by use of affinity HPLC analysis as previously taught herein. The level Three population uses array analysis on combinations of four fragments each containing grouping of active amino acids in six long amino acid fragments of the Level One ligand. The fragment combinations is defined by using combinations of the Level Two active amino acids plus three additional amino acids with second-order Chiral activity (Norleucine for Methionine, Tryptophan and Tyrosine) that occur in ten of the 27 positions. The degree of Chiral activity from 78 combinations of these four fragments is used to prescribe the sequence of the 27 amino acids ligand that is synthesized to be specific for the patient an represents one out of approximately 10,000 possible combinations.

According to still another feature of the invention the extract of the patient's GRP-r is capture in a trap containing suitable affinity chromatography media (the Trap) and the Level One ligand is: chemically tagged with fluorescence dye, contacted with the Trap and the presence of same measure in the discharge from the Trap. A reduction in the fluorescence in the discharge from the Trap greater than the expected dilution from chromatograpy shows that GRP-r is present in the Trap and bound to some quantity of the ligand and presumes that the patient has cancerous cells. This presumption of cancer is to be confirmed by other tests not within the scope of this invention.

According to another feature of the invention an electronic platform is mated to the above described array of fragments to rapidly determine the Chiral activity at each of the 78 probe points in relative electrical terms based on measuring the salt bridge as previously taught.

According to still another feature of the invention a PET scan on a patient is performed by administering a radiopharmaceutical with the highest Chiral activity made from radiological tagging one of the 10,000 Third Level ligands. This patient specific radiopharmaceutical delineates and quantifies the highly specific GRP-r cellular receptors molecular target and the endogenous ligand is the transporter in the radiopharmaceutical tracers.

In another feature of the invention the radioactive tagging to form radiopharmaceuticals is added at the PET scan location or point of use by organometallic chemistry without the need for a cyclotron that requires regionalize manufacture and a sophisticated distribution system due to the short shelf-life of the radiopharmaceutical.

Empty receptors bind to ligands as a result of Brownian encounters, forming new complexes with the frequency of binding proportional to the concentration of the GRP-r There is also a characteristic frequency with which existing ligand-receptor complexes dissociate as a result of thermal excitation. The equilibrium constant of disassociation Kd measured in molecules per nm³ is equal to k_{d}/kₐ where kd is the dissociation rate constant in sec⁻¹ and kₐ is the association constant in sec⁻¹. The kₐ rate constant is mainly affected by the molecular weight of the ligand. The k_{d} rate constant is effected by pH change to a much greater extent than is the kₐ rate constant. For example a pH change for 7.5 to 4.0 for growth hormone results in a 1,600 times change in the k_{d} rate constant and only a 1.7 times change in the ka rate constant. The receptor affinity is the inverse of the disassociation constant. The smaller the kd the greater the affinity and the more firmly the ligand grasps the receptor.

Affinity, Specificity and Chiral Activity:

While affinity measures the strength of the binding of a ligand probe to the GRP-r target, the specificity defines the degree to which a receptor can distinguish between similar ligands. The affinity of a ligand probe for the GRP-r must be greater than the affinity of any other ligand probe by some threshold multiple to allow the binding to occur. This multiple for single-residue affinity reduction at a receptor-accessible surface is of the order of 10⁻³.

The considerations for affinity and selectivity on a theoretical basis include: the GRP-r accessible surface, the amino acid sequence of the ligand, determination of the number and positions of the active sites, measurement of the hydrogen bonding between the ligand and the GRP-r and understanding the binding packets and the docking interactions.

The GRP-r is in a heptahelical structure where in the third extracellular domain of the receptor is the segment of the receptor where ligand attachment is the focus.

Receptor modeling shows that: 1. Threonine, Phenylalanine and Serine of this domain are the critical residues for determining GRP-r selectivity and suggest that both hydrogen bonding and receptor-ligand cation-pie interactions are important or their high affinity interactions, and 2. each of the three amino acids face inward and within 5 Angstroms of the putative binding pocket. The expressed GRP receptors on cells from small-cell lung cancer have a high affinity expressed by a K_{d} of 1.5 molecules per nm³ and a count of approximately 6,700 receptors per cell. The probability of occupancy of a ligand probe P_{occupied} is equal to (C_{GRP receptor}/K_{d}) P_{unoccupied}. To obtain a 90% probability of occupancy the K_{d} must be ten percent of the concentration of GRP: 0.1×C_{GRP receptor}. In the present invention the GRP receptor is separated from the tissue cells and concentrated so that a 90% probability of attachment with a ligand probe is obtained. This concentration is 15 molecules per nm3 based on extrapolation from the data for small-cell lung cancer cells.

Chirality is defined as the spin every molecule and cell receptor has naturally. Every molecule found in nature is Chiral, meaning it spins either to the left or the right. Chirality has been described as a geometric phenomenon that occurs when two molecules are identical in ever way, with the exception that they are mirror images of each other. In the present invention the concept of Chirality is expanded to be Chiral activity as relative specificity and affinity of one ligand for a particular GRP-r relative to another ligand wherein both ligands have the same number of amino acids but differ is sequence of Amino Acids.

Receptor sites are found on cell membranes and are similar to three dimensional locks on the doors of your home, thousands of receptors coat each cell and wait for the ligands—molecular keys—the most Chirally correct ligands have the greatest probability that they will fit the lock and thus poses the maximum Chiral activity. The ligands can be subdivided into fractions containing subsets of the ligand that are Chiral and deposited in arrays to be in contact with an extract of the patient's GRP-r. The relative Chiral activity of each probe is measure by the strength of the “salt bridge” established between the two proteins (ligand and GRP-r) and the associated signal is received on a electronic platform as taught herein.

Affinity Chromatography:

In affinity chromatography, a protein with a specific affinity for a ligand to be isolated is attached to a solid matrix typically through an amide linkage. In the present invention the matrix is an agarose-base where the carbohydrate nature of the media provides a chemically favorable environment for coupling the GRP-r and the high cross-linked structure of the media's spherical beads provides excellent chromatographic properties. The ligand is applied to the matrix-bound GRP-r and other components of the mixture that is applied with no affinity for the GRP-r are washed through and appear in the column discharge. A flurometer can be used to measure the presence and concentration of ligands in the column discharge when the ligand has been tagged with an amine-reactive fluorescent dye. By choosing the ligand-dye conjugation conditions in a buffer with pH selected below the pK values for the amino acids the dye can be attached to the amine terminus of the ligand and the mating of the ligand with the GRP-r undisturbed by maintaining the ligand in a non-protonate form.

Positron Emission Tomography:

Positron Emission Tomography (PET) is one the most commonly used modality in nuclear medicine and its efficacy in cancer detection and staging. PET differs from anatomically-based imaging modalities, such as MRI and X-rays, in that it assesses the level of metabolic activity and perfusion in various organ systems. The PET process produces biologic images based on the detection of gamma rays that are emitted by a low dose radioactive substance referred to as PET tracers: the radiopharmaceutical compound used in conjunction with a PET scan to enhance the imaging process. FDG (11C-deoxyglucose and 11C-methionine) is the most common PET tracer used today.¹ FDG is produced in a cyclotron and is tagged to glucose. FDG can help in locating a tumor, because the faster growing cancer cells absorb glucose faster than other “normal” tissues in the body. Other less used type of PET tracers are a class of radiophamaceuticals called Targeted tracers in which the present invention falls. Targeted tracers delineates and quantifies highly specific molecular targets, such as cellular receptors. These transporters are either endogenous ligands or drugs (e.g., 11C-raclopride for the DA2 dopamine receptor).

In oncology, PET in conjunction with a FDG tracer is the study of choice with approximately 90 percent of all PET procedures are performed for oncology purposes. However, although PET scans play a diagnostic role, its major contribution has been in the accurate staging of cancer treatment.

One limitation of the FDG is that the absorption for different tumor types and can miss small lesions (<1 cm). Thus, a negative scan does not prove that a patient is free of cancer. Another limitation is that FDG measures metabolic activity, PET studies have demonstrated that muscle activity can be detected by PET scans as false positives. PET and the use of FDG and other presently available radiopharmaceutical tracers already have provided the oncology imaging community with great diagnostic advances, but as also formulated in the National Cancer Institute 2005 plan, improvements are still needed in oncology imaging that will enhance the detection and diagnosis of cancerous cells.

### EXAMPLE 1

In the present invention a population 27 ligands plus the ligand in claim 1 is defined using combinations of the three active amino acids with first-order Chiral activity (Proline, Alanine and Arginine) that occur in six of the 27 positions. This population is reduced to a total population of nine by use of affinity HPLC analysis as taught herein. In the analysis extraction of the patient's GRP-r from prostate cancer tissue is performed by the steps of composite sampling, separation, enrichments, re-suspension and mining the active fragment. Tests are performed on 50 prostate cancer tumors from archival or biopsy specimens of 50 patients. Each sample is one milliliter of clear solid free liquid at a concentration of an estimated 300 micrograms per milliliter of GRP-r and is obtained from tissue samples of 1 grams wet weight of prostrate tissue. The steps in the preparation of the GRP-r are described in the following paragraphs.

Composite Tissue Sample:

Aliquots of surgically resected tumors or biopsy specimens submitted for diagnostic analysis are frozen immediately after surgical resection and stored at −70° C.

Cryostat sections (20-μ thick) of the tissue samples or cylindrical samples are removed by “core biopsy” from archival tissue specimens of a primary tumor. A 0.6 mm diameter, or smaller, core biopsy needle is used to keep wastage of the original sample to a minimum and retain morphological information.

Three core biopsies are taken from distant parts of the specimen.

Separation of GRP-r from Tissue:

Tissue samples are deparaffininized with three charges of xylene for two minutes each, followed by rehydration by exposing the specimen to successive two minute washes in graded ethanol (absolute, 95%, 70%, and 50%).

The specimen is then treated with proteinase k (5 ug/ml) in phosphate buffered saline (PBS) for 60 minutes at 37 degree C. in order to make the cells permeable.

Then the specimens are washed in PBS and then dehydrated in graded ethanol (50%, 70%, 95%, and absolute) for two minutes each.

Enrichment of GRP-r:

GRPr-enriched membranes are obtained as a post nuclear membrane P2 fraction from cells. The P2 fraction is obtained by washing the cells twice with 10 ml of phosphate-buffered saline (PBS) at room temperature and incubated at 4° C. for 15 min in 5 ml of solution A (10 mM Hepes, pH 7.4/1 mM EGTA) fortified with 100 μM 4-(2-Aminoethyl)-benzenesulfony; floride HCL (AEBSF purchased from ICN).

The swollen cells are harvested by scraping and homogenized in a Dounce homogenizer (15-20 strokes with the tight pestle), and the nuclei and cell debris are removed by centrifugation at 750× g for 10 min at 4° C.

The post nuclear membrane fraction P2 is collected from the supernatant by centrifugation at 75,000× g for 30 min at 4° C.

Re-Suspension:

The P2 membrane pellet is re-suspended in solution A containing a chaotropic agent (6 M urea), incubated on ice for 30 min and sedimented at 75,000× g for 30 min at 4° C.

After a second extraction and centrifugation, the membrane pellet is washed once with solution A alone.

The final pellet is re-suspended in solution A supplemented with 12% (wt/vol) sucrose, and aliquots are frozen and stored at 80° C.

Mining the Active Fragment

The expected concentration is quantified based on a reference for GRP-r concentration in medullary thyroid carcinoma (MTC) tissue samples. This study reports that normal thyroid tissue contained less than 61 pmol GRP-r per gram wet weight; in contrast GRP-r concentration was elevated to as high as 7800 pmol/g in 32/34 tumor extracts. The concentration of GRP-r is 337 micrograms per milliliter assuming a sample consisting of one gram of tumor with a GRP-r concentration of 7,800 picomoles per gram is suspended in one ml of solute. The current study specified that 1 gram wet weight of prostate cancer tissue is to be extracted to obtain the required concentration of GRP-r for analysis.

### EXAMPLE 2

In the present invention an extract of the patient's GRP-r is captured in a trap containing suitable affinity chromatography media (the Trap) and the Level One ligand is: chemically tagged with fluorescence dye, contacted with the Trap and the presence of same measure in the discharge from the Trap. A reduction in the fluorescence in the discharge from the Trap greater than the expected dilution shows that GRP-r is present in the Trap and bound to some quantity of the ligand and presumes that the patient has cancerous cells. This presumption of cancer is to be confirmed by other tests not within the scope of this invention. The equipment used and the methods of analysis are described in the following paragraphs.

The equipment and major chemicals used are:

Fluorometer—Turner PicoFluor model number 80003 with mini cell adapter and cells model 8000-931; Fluorescent Dye—Molecular Probes AlexaFluor 488 a TFP ester model number A30005; Affinity Trap—Amershampharmaciabiotech custom HiTrap, 1 ml, with 10% by weight NHS-activated; Dye Separation Column—Amershampharmaciabiotech custom HiTrap, 1 ml, with G-10 Sephadex G-10: and Synthetic Ligand—Abgent synthetic peptide, >95% purity, synthesis ID# 5050421.

All buffers, solutions and contact times are in accordance with the manufactures suggested methods and procedures. A stock solution of the florescent dye is prepared. One drop (50 mico liters (ul) ) of the stock solution is placed in the 2 millileter (ml) bottle that contains 100 micro grams (ug) of the ligand. One drop of another solution is added to end the reaction. A slightly basic buffer is added to bring the volume to 1 ml. The top of the Dye Separation column is removed and replaced by a female luer fiting and the discharge end at the bottom is twist off to allow flow. The solution is fed by a syringe through the dye separation column and the discharge from the column is collected. The excess dye which has a characteristic low molecular weight is retained in the column. The top of the Affinity Column is removed and a drop of 1 mM HCL is added, a female luer fiting installed and the discharge end at the bottom is twisted off to allow ischarge. Six mls of 1 mM HCL is passed through the column to activate the media. The flow through the column is maintained at approximately 10 drops per minute. One ml of the patients GRP-r prepared in accordance with the method in Example 1 is passed through the Affinity Column and allowed to bond to the media. The media is then deactivated by passing a sequence of buffers in 6 ml dosges through the column: first high pH, then low pH and finally high pH again. The fluorescence tagged ligand is placed in the micro curvet with minimum volume of 40 ul and maximum volum of 200 ul and the florescence is measured. The solution is activated at 475=/−15 nm and fluorescence detected at 515+/−10 nm. One ml of the fluorescence tagged ligand is passed through the Affinity column followed by 1 ml of buffer solution and the discharge collected in two 1 ml aliquant. Representative samples of the two aliquant are placed in the micro curvet and the florescence is measured. The reduction or lack of presence of fluorescence in the samples of the aliquant is initiative that cancer maybe present in the patients clinical sample that was extracted for GRP-r.

### EXAMPLE 3

In the present invention radioactive tagging to form radiopharmaceuticals is added at the PET scan location or point of use by organometallic chemistry without the need for a cyclotron. The method used is described in the following paragraph.

The organometallic aquaion [^{99m}Tc(H₂O)₃(CO)₃]⁺ is used as a radiosynthon for the labeling of the bioactive ligand molecule for use in PET scans. The NH₂ terminus of the 27 mer ligand is functionalized to achieve radiolabeling by forming a high specific activity radiocomplex while maintaining the biological activity of the ligand. The aquaion is stable over a wide range of pH values and is characterized by excellent labeling efficiency. The labeling efficiency is associated with the presence on the three water molecules coordinated to thefac-M(CO)₃ which is characteristic of the aquaion not only the amine donor group in the present invention but also with thiols, phosphines and thioesters as donor groups.

Any enumerated listing of items does not imply that any or all of the items are mutually exclusive, unless expressly specified otherwise. The terms “a”, “an” and “the” mean “one or more”, unless expressly specified otherwise.

Although the present invention has been described chiefly in terms of the presently preferred embodiment, it is to be understood that the disclosure is not to be interpreted as limiting. Various alterations and modifications will no doubt become apparent to those skilled in the art after having read the above disclosure. Such modifications may involve other compositions or processes which are already known subsequently become known to be effective, and which may be used instead of or in addition to those already described herein. The compositions identified herein are not limiting but instructive of the embodiment of the invention, and variations which are readily derived or which are standard or known to the appropriate art are not excluded by omission. Accordingly, it is intended that the appended claims are interpreted as covering all alterations and modifications that fall within the true spirit and scope of the invention in light of the prior art.

Additionally, although claims have been formulated in this application to particular combinations of elements, it should be understood that the scope of the disclosure of the present application also includes any single novel element or any novel combination of elements disclosed herein, either explicitly or implicitly, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

All of the publications, patents, and patent applications referenced above in the provisional application are herein incorporated by reference in their entireties to the same extent as if each individual publication, patent, patent application, or provisional patent application was specifically and individually indicated to be incorporated by reference in its entirety.

What is claimed is:

1. A process for the production of a recombinant patient specific tumor receptor ligand, useful in cancer diagnostic and therapeutic applications comprising:
identifying a receptor type present on a cancer cell obtained from a host;
identifying a ligand to that type receptor which ligand is indigenous to said host and which has an ability to bind to said receptor;
providing a plurality of tumor receptor specific ligands associated with said host indigenous ligand, wherein said tumor receptor specific ligands exhibit varying degrees of specificity and/or affinity for aberrantly expressed species of said receptor present on said cancer cell;
deriving a subset of patient specific tumor receptor ligands from said tumor receptor specific ligands; and
deriving or determining a recombinant patient specific tumor receptor ligand;
wherein said recombinant ligand is characterized as exhibiting the highest relative specificity and/or affinity with respect to said subset of patient specific tumor receptor ligands;
whereby providing a ligand equal to said recombinant patient specific tumor receptor ligand is useful in diagnostic and therapeutic applications associated with cancer.

2. The process of claim 1 wherein said receptor type is selected from the group consisting of Epidermal Growth Factor (EGF), Vascular Endothelial Growth Factor (VEGF), Gastrin Releasing Peptide Receptor (GRP-r), or Neuromedin B.

3. The process of claim 1 wherein said receptor is GRP-R.

4. The process of claim 1 wherein said recombinant patient specific tumor receptor ligand is produced by a method selected from the group consisting of protein sequencing, genetic engineering, or mining of a library containing the endogenous end of bacterial phage PIII and PVI proteins.

5. A recombinant patient specific tumor receptor ligand produced in accordance with the process of claim 1.

6. A recombinant patient specific tumor receptor ligand produced in accordance with the process of claim 4.

7. The recombinant patient specific tumor receptor ligand of claim 5 further including at least one radiologic isotope tag selected from the group consisting of 99-Mo (technetium Tc99m), 90-y, 111-In, 123-I, 186-Re, 32-P, 81m-Kr, 89-Sr, 103-Pd, 117m-Sn, 131-I, 47-Sc, 62-Zn, 64-Cu, 68-Ge, 153-Gd, 166-Ho or 177-Lu.

8. The recombinant patient specific tumor recept6r ligand of claim 5 further including at least one chemotherapeutic agent selected from the group consisting of cyclophosphamide, mechlorethamine, mephalin, chlorambucil, heamethylmelamine, thiotepa, busulfan, carmustine, lomustine, semustine, methotrexate, fluorouracil, floxuridine, cytarabine, 6-mercaptopurine, thioguanine, pentostatin, vincristine, vinblastine, vindesine, etoposide, etoposide orthoquinone, and teniposide, daunorubicin, doxorubicin, mitoxantrone, bisanthrene, actinomycin D, plicamycin, puromycin, and gramicidine D, paclitaxel, colchicine, cytochalasin B, emetine, maytansine, and amsacrine, aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, fioxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, or zoledronate.

9. A process for the production of a recombinant patient specific tumor receptor ligand, useful in cancer diagnostic and therapeutic applications comprising:
providing a serum or tissue sample obtained from a host and suspected of containing cancerous cells;
providing a plurality of species of ligands to plural types of receptors, which ligands species are indigenous to said host, and wherein each of said species is known to bind to a receptor found on a cancer cell;
confirming binding of at least one of said species of host indigenous ligands to a cancer cell;
providing a plurality of tumor receptor specific ligands associated with said bound indigenous ligand, wherein said tumor receptor specific ligands exhibit varying degrees of specificity and/or affinity for aberrantly expressed species of said receptor present on said cancer cell;
deriving a subset of patient specific tumor receptor ligands from said tumor receptor specific ligands; and
deriving or determining a recombinant patient specific tumor receptor ligand;
wherein said recombinant ligand is characterized as exhibiting the highest relative specificity and/or affinity with respect to said subset of patient specific tumor receptor ligands; whereby providing a ligand equal to said recombinant patient specific tumor receptor ligand is useful in diagnostic and therapeutic applications associated with cancer.

10. The process of claim 9 wherein said receptor type is selected from the group consisting of Epidermal Growth Factor (EGF), Vascular Endothelial Growth Factor (VEGF), Gastrin Releasing Peptide Receptor (GRP-r), or Neuromedin B.

11. The process of claim 9 wherein said receptor is GRP-R.

12. The process of claim 9 wherein said recombinant patient specific tumor receptor ligand is produced by a method selected from the group consisting of protein sequencing, genetic engineering, or mining of a library containing the endogenous end of bacterial phage PIII and PVI proteins.

13. A recombinant patient specific tumor receptor ligand produced in accordance with the process of claim 9.

14. A recombinant patient specific tumor receptor ligand produced in accordance with the process of claim 12.

15. The recombinant patient specific tumor receptor ligand of claim 13 further including at least one radiologic isotope tag selected from the group consisting of 99-Mo (technetium Tc99m), 90-y, 111-In, 123-I, 186-Re, 32-P, 81m-Kr, 89-Sr, 103-Pd, 117m-Sn, 131-I, 47-Sc, 62-Zn, 64-Cu, 68-Ge, 153-Gd, 166-Ho or 177-Lu.

16. The recombinant patient specific tumor receptor ligand of claim 13 further including at least one chemotherapeutic agent selected from the group consisting of cyclophosphamide, mechlorethamine, mephalin, chlorambucil, heamethylmelamine, thiotepa, busulfan, carmustine, lomustine, semustine, methotrexate, fluorouracil, floxuridine, cytarabine, 6-mercaptopurine, thioguanine, pentostatin, vincristine, vinblastine, vindesine, etoposide, etoposide orthoquinone, and teniposide, daunorubicin, doxorubicin, mitoxantrone, bisanthrene, actinomycin D, plicamycin, puromycin, and gramicidine D, paclitaxel, colchicine, cytochalasin B, emetine, maytansine, and amsacrine, aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, or zoledronate.
